(19) United States
(12) Patent Application Publication
(10) Pub. No.: US 2026/0206888 A1
(43) Pub. Date: Jul. 23, 2026

(54) Multi-Layered Polymer Product With Single Don Function

(71) Applicant: WRP Asia Pacific Sdn. Bhd., Sepang (MY)

(72) Inventors: Bee Hong Goh, Negeri Sembilan (MY); Letchemy Muthu, Negeri Sembilan (MY); Geok Chin Ng, Selangor (MY); Nadarajah Rajah Swaminathan, Negeri Sembilan (MY); Wai Leong Leong, Selangor (MY); Mahaletchumy Therumoorthe, Selangor (MY); Muhamad Shahril Mazwan Mazlam, Kelantan (MY); Nur Afifah Farhana Roslan, Negeri Sembilan (MY)

(73) Assignee: WRP Asia Pacific Sdn. Bhd., Sepang (MY)

(21) Appl. No.: 19/143,604

(22) PCT Filed: Mar. 31, 2023
(86) PCT No.: PCT/MY2023/050021

§ 371 Date: Jun. 26, 2025

(30) Foreign Application Priority Data
Dec. 27, 2022 (MY) PL2022007428

(51) Int. Cl. A41D 19/00; A42B 1/12; A61F 6/04; B29C 41/14; B29C 41/22; B29C 41/46; B29K 9/00; B29K 105/00; B29L 31/48

(52) U.S. Cl. CPC A41D 19/0006; A41D 19/0058; A42B 1/12; A61F 6/04; B29C 41/14; B29C 41/22; B29C 41/46; B29K 2009/00; B29K 2105/0064; B29L 2031/4864

(57) ABSTRACT

The present invention relates to a multi-layered polymer product (1) in a single don, that comprises, a main body (4) and a secondary body (5), characterized in that the main body (4) and secondary body (5) have an outer, an intermediate layer comprising an anti-tack solution coating and a polymer coating, and an inner layer (3). The manufacturing process of the multi-layered polymer product (1) is also disclosed to be a dipping process that is made up of three latex layers.

### FIELD OF INVENTION

The present invention relates to a multi-layered polymer product with a single don function and the method of producing the same.

### BACKGROUND OF THE INVENTION

Since the discovery of latex, the creation of many new and conventional products has been rampant throughout the years. By being flexible, durable, and waterproof, these products have brought about comfort and convenience to our everyday life. One good example of such a product would be gloves, whereby to-date, there are many types of gloves being made from latex, such as examination gloves, surgical gloves, industrial gloves, household gloves and many more. Furthermore, with low-cost and high production yield, it is no surprise that the glove making industry, especially disposable surgical gloves, has been on the rise.

The demand for disposable surgical gloves has increased significantly in recent years, especially during the current pandemic. Used in many different industries such as health care, research facilities, or cleaning services, gloves are worn by many to protect themselves from direct skin contact with any harmful or infectious substances that they might encounter during the course of their work. The demanding nature of these types of jobs require disposable gloves to be durable but flexible, provide a good grip, while at the same time not limiting the dexterity and movement of the fingers when worn. Moreover, these gloves should also be hypoallergenic as to not cause any irritation and depending on the nature of the usage, sterile as well. Due to the need of these disposable surgical gloves being flexible while still maintaining the sense of touch, it is no surprise that the thickness of gloves is an important factor.

Therefore, to reduce the chances of transmission and increase safety, it is a common practice for glove users, especially those in the medical line, to don two gloves, one over the other to prevent infections or seepage of harmful substances due to undetected perforation of gloves. While this method does help to prevent transmission, other problems have been identified, such as slipping of the outer layer of glove, or lesser sense of touch due to double glove thickness. These limitations are particularly concerning during certain activities, such as in surgery.

Another product of interest would be the manufacturing of condoms, and similar to that of surgical gloves mentioned above, condoms are manufactured to be as thin as possible in order to allow users to not lose any sense of touch, while at the same time being durable to ensure that breakage does not occur.

Furthermore, as mentioned earlier, high demand has driven high production yield, therefore, one of the main challenges that glove and condom manufacturers must overcome would be to reduce the percentage of defects during production, mainly hole defects. Unfortunately, most of the time, the presence of holes is not easily detected, and could pose a hazard risk to the user, especially when dealing with infectious or dangerous substances. Moreover, apart from manufacturing defects, these holes can also occur accidentally due to scalpel or needle damage by the user.

To-date, there have been attempts address these problems. Australian patent AU2018212491B2 discloses a disposable multi-layer glove, connected at the fingertips and cuff region, that comprises of an outer layer, an inner layer and an intermediate layer which is made up of particles having a mean diameter of ≤100 μm and a surface comprising exposed OH groups. Moreover, the manufacturing process of this invention involves a dipping process of two latex layer with a final glove thickness of 100 μm to 800 μm.

US application no. US2006026737A1 relates to a multi-layered glove comprising an inner layer and an outer layer with pigments between these layers to act as a breach indicator. The presence of a breach indicator alerts the user of a tear in the glove which may prevent transmission of infection. However, the thickness of the final product is not specified nor disclosed and may present further limitations as thickness plays an important part in allowing the user to maintain his or her sense of touch to allow better working conditions.

EP1961319A1 on the other hand further discloses a multi-layer polymer glove, which is made up of triple layers at the fingertips, double layer at the palm region and a single layer at the wrist region and is intended to protect the user from cold and chemical spills. However, due to the fact that the glove has three layers at the fingertip region, the sense of touch is significantly reduced. Overall, while the glove may provide good durability, the flexibility is hindered.

KR100190485B1 meanwhile discloses a multi-layered condom which can prevent the leakage of internally generated fluid such as semen, or the penetration of foreign substances which could be harmful to the user. The invention consists of a peelable outer surface which prevents the outer layer from contacting with the inner surface of the condom is being used. While this invention allows better protection due to it being multi-layered, it doesn't provide for a way to detect any breach of fluid, which could be helpful to prevent any unwanted transmission.

Hence, there is a requirement for a new polymer product that provides users with protection equivalent to two gloves in a single don which is not only durable, but also thin and flexible enough and that does not affect the sense of touch of the user.

### SUMMARY OF INVENTION

The objective of the present invention is to produce a new polymer product that provides users with protection equivalent to two gloves in a single don which is not only durable, but also thin and flexible enough and that does not affect the sense of touch of the user, as well as a method of producing thereof.

The present invention relates to a multi-layered polymer product comprising a main body and a secondary body, characterized in that said main body and secondary body comprises:
i. an outer layer;
ii. one or a plurality of intermediate layers; and,
iii. an inner layer,
wherein the outer, intermediate, and inner layers of the main body are attached at a region as to form discrete layers, while the outer, intermediate, and inner layers of the secondary body are attached together entirely.

The intermediate layers of the main body comprise an anti-tack solution coating and a polymer coating.

The anti-tack solution coating of the present invention comprises of:
i. an anti-caking agent;
ii. a pearlescent agent;
iii. a conditioning agent;
iv. a surfactant;
v. a functional lipid; and,
vi. polyglyceryl ester.

The polymer coating of the present invention comprises of:
i. a binder;
ii. a stabilizer;
iii. a silicone;
iv. a wax emulsion;
v. a matting agent;
vi. a dispersing agent;
vii. a wetting agent; and,
viii. a coupling agent.

The present invention further comprises a method for producing a multi-layered polymer product comprising the steps of:
i. providing a former having in a shape of a desired product;
ii. dipping said former in a first coagulant, to a preset immersion depth t₁;
iii. dipping said former in a first latex dispersion to the immersion depth t₁ to produce an inner layer;
iv. dipping said former in a second latex dispersion to the immersion depth t₁ to produce at least one intermediate layer;
v. dipping said former in a polymer coating to a preset immersion depth t₂;
vi. dipping said former in an anti-tack solution coating to the immersion depth t₂,
vii. dipping said former in a second coagulant to the immersion depth t₁
viii. dipping said former in the first latex layer to the immersion depth t₁ to produce an outer layer,
ix. dipping said former in a second polymer coating to a preset immersion depth t₃.

Also provided is a vulcanization step for the outer, intermediate, and inner latex layers carried out at a temperature between 80° C. to 120° C., preferably at 100° C.

Furthermore, the present invention also relates to a multi-layered polymer product which can either be of a glove, condom, raincoat, shower cap or any polymer products that requires donning, and the method of producing thereof.

### BRIEF DESCRIPTION OF DRAWINGS

FIG. 1 shows a multi-layered polymer product with its specific layers.

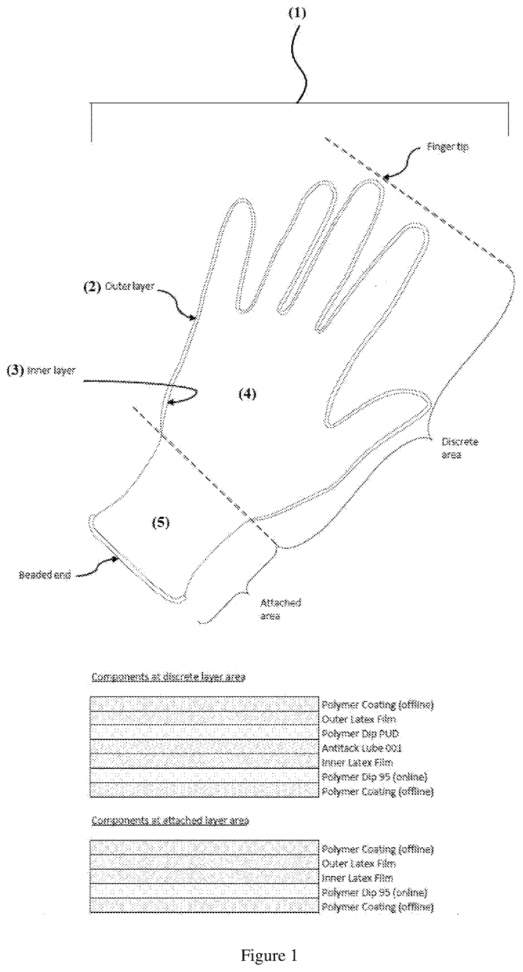

FIG. 2A illustrates a manufacturing process of the multi-layered polymer product.

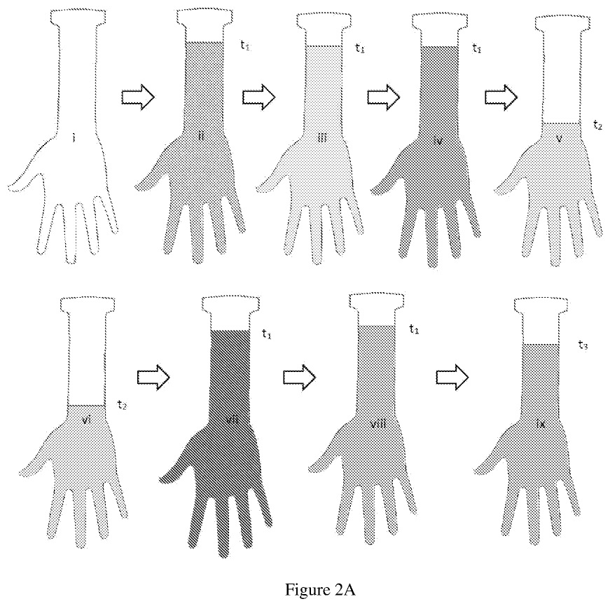

FIG. 2B describes the individual stages of the manufacturing process of the multi-layered polymer product.

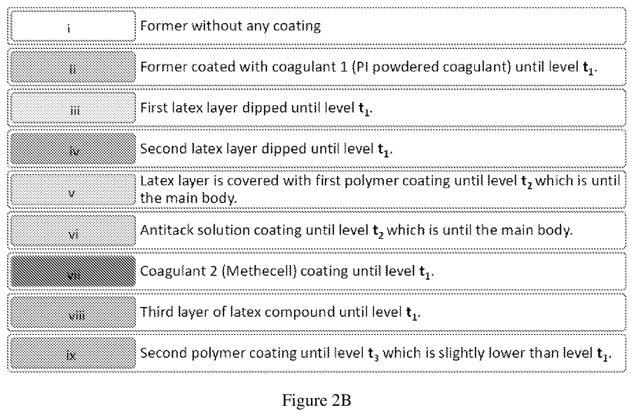

FIG. 2C provides a flowchart of the three latex layer process in the manufacturing of the multi-layered polymer product.

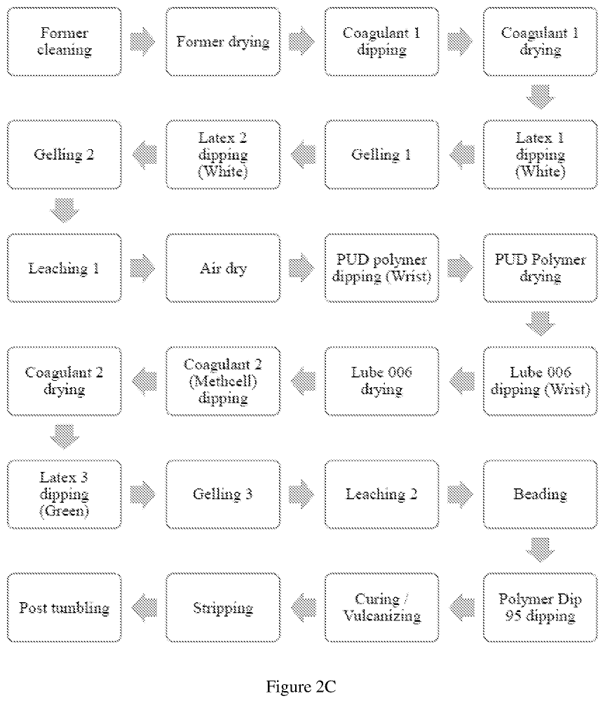

### DETAILED DESCRIPTION

The present invention relates to a multi-layered polymer product (1) comprising a main body (4) and a secondary body (5), characterized in that said main body (4) and secondary body (5) comprise an outer layer (2), one or a plurality of intermediate layers and an inner layer (3).

The present embodiment of the multi-layered polymer product is a glove. However, it will be understood by a person skilled in the art that the multi-layered product can be applied to other products, such as a condom, raincoat, shower cap, or any other suitable polymer article.

In the present embodiment, the present invention also provides for the outer layer to have a surface with improved and consistent grip properties, and for the inner layer to have an inner surface with easy donning on and donning off properties.

In an embodiment of the present invention, the outer (2), intermediate, and inner (3) layers of the main body (4) are attached only at a region of the main body (4), with the remaining layers being discrete from each other. It is appreciated that the attached region can be any part of the main body such as the tip or the end, but not limited to one region only.

In this particular embodiment, the outer, intermediate, and inner layers are attached at the secondary body (5) (not pictured). It will be appreciated that although the layers of the secondary body in this embodiment are attached together entirely, partial adhering of layers (such as at a section or region) of the secondary body may be utilized in other embodiments.

Further, in the present embodiment, the intermediate layer of the main body (4) comprises an anti-tack solution coating and polymer coating, which the anti-tack solution coating comprises
i. an anti-caking agent;
ii. a pearlescent agent;
iii. a conditioning agent;
iv. a surfactant;
v. a functional lipid; and,
vi. polyglyceryl ester.

In one embodiment of the present invention, said anti-caking agent comprises soap, detergent stearate or a combination thereof, which functions as a separating agent to allow the separation between the discrete layers of the polymers, while said conditioning agent is an emulsion that provides a smooth finishing on the latex layer. In another embodiment of the present invention, the emulsion can be a cationic emulsion of an amine-functional silicone polymer, but not limited to.

Furthermore, in one embodiment of the present invention, said surfactant can be anionic, cationic or a combination thereof which helps to condition the surface between the discrete layers.

In one embodiment of the present invention, said polymer coating of the intermediate layer comprises
i. a binder;
ii. a stabilizer;
iii. a silicone;
iv. a wax emulsion;
v. a matting agent;
vi. a dispersing agent;
vii. a wetting agent; and,
viii. a coupling agent.

In the same embodiment of the present invention, the binder comprises a soft binder, an acrylic binder, a medium hard binder, a styrene acrylic binder or a combination thereof, which functions to smoothen the surface of the product, reduce the friction between the polymer layers as well as to hold all materials in the acrylic polymer coating together. The silicone can be of low molecular weight, high molecular weight, or a combination thereof which helps to reduce coefficient friction and helps in layer separation.

The presence of the above anti-tack coating solution and the polymer coating facilitates in the production of discrete layers, as these two coatings prevent the adhering of polymer layers during the manufacturing process.

In an embodiment of the present invention, the multi-layered polymer product (1) comprises an elastomer selected from polyisoprene-polyisoprene latex, polyisoprene-chloroprene latex, polyisoprene-nitrile butadiene latex, polyisoprene-chloroprene-nitrile butadiene latex, or a combination thereof, comprising a thickness of 0.5 mm to 1.5 mm at the main body (4), while the secondary body (5) comprises a thickness of more than 0.3 mm. In addition, the secondary body is at least 50% thicker than a product with a single polymer layer, thus having better tear strength and rupture resistance. Despite being multi-layered, the polymer product is thin and does not restrict movement or dexterity when worn.

In an embodiment of the present invention, the outer layer (2) and inner layer (3) of the main body (4) may be different in color to function as a breach indictor.

In alternative embodiments of the present invention, said product comprises a glove, bag, condom, or shower cap, but is not limited to. In another embodiment of the present invention, the product can also be any polymer products that requires donning.

In another embodiment of the present invention, the method of producing a multi-layered polymer product comprises the steps of:
i. providing a former having a shape of a desired product;
ii. dipping said former in a first coagulant to a preset immersion depth t₁;
iii. dipping said former in a first latex dispersion to the immersion depth t₁ to produce an inner layer;
iv. dipping said former in a second latex dispersion to the immersion depth t₁ to produce at least one intermediate layer;
v. dipping said former in a polymer coating to a preset immersion depth t₂;
vi. dipping said former in an anti-tack solution coating to the immersion depth t₂;
vii. dipping said former in a second coagulant to the immersion depth t₁;
viii. dipping said former in the first latex layer to the immersion depth t₁ to produce an outer layer,
ix. dipping said former in a second polymer coating to a preset immersion depth t₃.

In this particular embodiment of the present invention, the preset immersion depth t₁ covers both the main body (4) and secondary body (5), while the preset immersion depth t₂ covers only the main body (4). The preset immersion depth t₃ on the other hand covers both the main body (4) and secondary body (5), but is shallower than the depth of t₁ at approximately 2 cm to 8 cm shorter than immersion depth t₁.

In the same embodiment of the present invention, the dipping of the first and second latex layer together with the anti-tack solution coating and polymer coating will result in discrete layers allowing for better breach detection. Furthermore, with each latex layer having different colors, breach detection is further enhanced.

The method as disclosed above may be utilized to produce said multi-layered polymer product (1) as described previously.

In an embodiment of the present invention, a vulcanization step is carried out at a temperature between 80° C. to 120° C., preferably 100° C., on said outer (2), intermediate and inner (3) latex layers.

In one embodiment, the present invention also relates to a multi-layered polymer product which can either comprise a glove, raincoat, condom, shower cap or any polymer products that requires donning, and a method of production thereof.

In another embodiment of the present invention, the polymer coating used in the method of the present invention comprises
i. a binder;
ii. a stabilizer;
iii. a silicone;
iv. a wax emulsion;
v. a matting agent;
vi. a dispersing agent;
vii. a wetting agent; and,
viii. a coupling agent.

### Examples of Multi-Layered Polymer Products

### Example 1

In one example, the product of the present invention is a surgical glove. One important property of a good pair of surgical gloves is to have a comfortable feel while doing any activities. Moreover, a glove which is multi-layered provides the user with the ability to wear multiple-layered gloves in a one don system. Advantageously, the cuff of the glove will not slip or roll down during use, since exposing the user's hand to chemicals or biological fluids may be harmful.

Multiple gloving, such as double gloving as described above, reduces risk of inner glove perforations by 71% compared to single gloving and are recommended by professional organizations such as Association of periOperative Registered Nurse (AORN), World Health Organization (WHO) and Centers for Disease Control and Prevention (CDC).

Moreover, in this example of the present invention, the discrete layers are in different colors, thus allowing earlier detection of a glove seepage, so that the user can change their gloves once they realize the glove has been perforated. Once a perforation is detected or noticed, the glove can be removed quickly as the multi-layered gloves are connected at the cuff such that it is donned as a single glove and similarly replaced in a single action despite being multi-layered. This also saves time and effort as compared to wearing 2 pieces of gloves. It is interesting to note that 82% of perforations of the outer glove go unnoticed by those who double glove, posing a risk to the glove wearer.

Therefore, to achieve this, in one embodiment of the present invention, but not limited to, the product is designed into a glove shape by using a conventional straight finger former in the shape of a hand which enables multiple layers to form and be of the same size without any bagginess contouring the shape of the hand. These conventional designed straight finger formers are able to produce surgical gloves that have ideal fit with precisely crafted dimension in all areas making the user unaware of its presence during surgery and thus providing excellent dexterity.

### Example 2

### Composition of a Multi-Layered Polymer Product (1)

The tables below detail out the composition of each step in the production of a multi-layered polymer glove according to the present invention. It is understood that below is just one example of a possible composition used in the production of a polymer product, and is it not limited to only what is listed below. For example, a person skilled in the art will therefore understand that there exists different types or combination of polyisoprene latex which could also be used to produce the present invention. For example, while the below example states polyisoprene latex as the latex of choice, it is understood that other latex such as polyisoprene-chloroprene latex, polyisoprene-nitrile butadiene latex or a combination can also easily replaced as the latex instead.

TABLE A
Synthetic Latex Compound

| | Part by | | | |
|---|---|---|---|---|
| No. | weight, % | Material | Functionality | Examples of Material |
| 1. | 85-95 | Synthetic latex | A colloidal dispersion | Polyisoprene, Polychloroprene, Nitrile, |
| | | | of a polymeric material | Thermoplastic elastomer |
| | | | in liquid system. | |
| 2. | 0.4-0.6 | Stabilizer | Maintain colloidal | Ammonia, Potassium Hydroxide, SDBS |
| | | | properties | |
| 3. | 2.0-2.5 | Vulcanizing agent | Allow crosslinking of | DPTT, Sulphur, Accelerator Free Crosslinker |
| | | | polymers | |
| 4. | 2.5-3.0 | Activator | Aid in crosslinking of | Zinc Oxide |
| | | | polymers | |
| 5. | 0.4-0.8 | Accelerator | Control rate of | ZDEC, ZMBT, ZDBC, DIXP, SDBC |
| | | | vulcanization | |
| 6. | 0.8-1.0 | Antioxidant | Impart age resisting | Wingstay L/ Ralox LC/ Ionol LC |
| | | | properties to finished | |
| | | | products | |
| 7. | 0-0.4 | Pigment | Impart colour to the | Titanium Dioxide, Brown, Green, Yellow |
| | | | finished product | |

TABLE B
First Coagulant Compound

| No. | Concentration, % | Material | Functionality | Examples of Material |
|---|---|---|---|---|
| 1. | 14.0-20.0 | Coagulating agent | Coagulate liquid latex | Calcium Nitrate |
| | | | around the former to | |
| | | | form wet gel film | |
| 2. | 3.0-8.0 | Releasing agent | Ease cured glove | Calcium carbonate |
| | | | stripping | |

TABLE C
Second Coagulant

| No. | Concentration, % | Material | Functionality | Examples of Material |
|---|---|---|---|---|
| 1. | 14.0-15.0 | Coagulating Agent | Coagulate liquid latex around | Calcium Nitrate |
| | | | the former to form wet gel film | |
| 2. | 70-90 | Solvent | Aid in drying coagulant quicker | Isopropanol Alcohol, Methanol, Ethanol |
| 3. | 3.0-5.0 | Thickener | Increase solution viscosity to | Metholose, Xanthan Gum, HPMC, HEC |
| | | | reduce dripping and flow back | |

TABLE D
First On-line Polymer Compound (Dip PUD)

| | Parts by | | | |
|---|---|---|---|---|
| No. | weight, % | Material | Functionality | Examples of Material |
| 1. | 40-50 | Aqueous anionic | Provide high abrasion | PUD (Leasys 5530, 3102, 3900, 5531, 3459, |
| | | dispersion | resistance, gloss and | 3468 / Archsol 8407, 8355) |
| | | | flex performance | |
| 2. | 15-25 | Styrene acrylic | Holds all materials in | Nitrile latex, DT-77, DT-617, DT-6034, |
| | | binder | the polymer together | E1383, DT-6191 |
| 3. | 1.0-2.0 | Non-ionic stabilizer | Help to lower surface | Agwet 100N, Triton X-100, Teric 320, |
| | | | tension of chemicals in | Tergitol 15-S-9 |
| | | | the polymer | |
| 4. | 12.0-14.0 | High molecular | Reduce coefficient | DC51, DC 23N, DC 402LS, Silcoglide T51 |
| | | weight silicone | friction and impart | |
| | | | gloss on the product | |
| | | | surface | |
| 5. | 3.0-4.0 | Microcrystalline | Provide lubrication | Bramul PA-35, Taurus 3971, |
| | | Wax emulsion | | Emustar-0001, Emustar -042X, |
| | | | | Michemlube 124 |
| 6. | 2.0-3.0 | Low molecular | Reduce coefficient | Xiameter MEM 0039, MEM 0349, |
| | | weight silicone | friction and impart | MEM 1785, MEM1171 |
| | | | gloss on the product | |
| | | | surface | |
| 7. | 9.0-10.0 | Matting agent | Provide matting effect | Pergopak M, M2, M3, M4, M5, M6, |
| | | | on the glove surface | Fume silica, Calcium Carbonate, |
| | | | | Kaolin clay |
| 8. | 0.10-0.30 | Dispersing agent | Disperse Pergopak | DisperBYK 192, Vultamol, Alginate, |
| | | | powder in the polymer | Lysurf GL-320A |
| | | | solution | |
| 9. | 3.0-4.0 | Wetting agent | Reduces surface | Silsurf A008/ACU 619, Teric 320, |
| | | | tension and reduces | Triton X-100, Surfynol 465, surfynol 440 |
| | | | foaming | |
| 10. | 2.0-3.0 | Solvent/coupling | offers moderate | Acrosolv DPM, Hengda-E8133, M8253, |
| | | agent | evaporation rate, high | DPMI, EP-g-MA |
| | | | dilution rate and has | |
| | | | very good coupling | |
| | | | abilities | |

TABLE E
Second On-line Polymer Compound (Dip 95)

| | Parts by | | | |
|---|---|---|---|---|
| No. | weight, % | Material | Functionality | Examples of Material |
| 1. | 48-75 | Acrylic/ Styrene | Main surface coating to | Neocryl XK 332, Neocryl XK 240, |
| | | acrylic binder | smoothen glove surface, | Nitrile latex, E1383 |
| | | | reduce friction between | |
| | | | glove surface and skin | |
| | | | and holds all materials | |
| | | | in the polymer together | |
| 2. | 1.0-2.0 | Non-ionic stabilizer | Help to lower surface | Agwet 100N, Triton X-100, Teric 320, |
| | | | tension of chemicals in | Teric 340 |
| | | | the polymer | |
| 3. | 11.0-15.0 | High molecular | Reduce coefficient | DC 51, Silcoglide T51, DC 23N, |
| | | weight silicone | friction and impart gloss | DC 402LS |
| | | | on the product surface | |
| 4. | 2.5-3.5 | Microcrystalline | Provide lubrication | Bramul PA-35, Taurus 3971, Emustar -0001, |
| | | wax emulsion | | Emustar - 042X, Michemlube 124 |
| 5. | 2.0-3.0 | Silicone emulsion | Reduce coefficient of | Xiameter MEM 0039, MEM 0039, |
| | | | friction and impart gloss | MEM 0349, MEM 1785, MEM1171 |
| | | | on the product surface | |
| 6. | 8.5-10.5 | Matting agent | Provide matting effect | Pergopak M, M2, M3, M4, M5, M6, |
| | | | on the glove surface | Fume silica, Calcium Carbonate, |
| | | | | Kaolin Clay |
| 7. | 0.15-0.30 | Dispersing agent | Disperse Pergopak | DisperBYK 192, Vultamol, Alginate, |
| | | | powder in the polymer | Lysurf GL-320A |
| | | | solution | |
| 8. | 3.0-4.0 | Wetting agent | Reduces surface tension | Silsurf A008/ACU 619, Teric 320, |
| | | | and reduces foaming | Triton X-100, Surfynol 465, |
| | | | | surfynol 440 |
| 9. | 1.0-2.0 | Solvent/coupling | offers moderate | Acrosolv DPM, Hengda-E8133, |
| | | agent | evaporation rate, high | M8253, DPMI, EP-g-MA |
| | | | dilution rate and has | |
| | | | very good coupling | |
| | | | abilities | |

TABLE F
Anti-tack Solution Coating - Lube 001/Lube006

| | Parts by | | | |
|---|---|---|---|---|
| No. | weight, % | Material | Functionality | Examples of Material |
| 1. | 70-90 | Metallic soap act as | Impart separating | Palmstar CAD 50C, Akstab LK 910, |
| | | separating | property in anti-tack | Akstab TBLM, Ligastar ca 860, |
| | | agent/anti-caking | solution coating to | Ligastar Li 600 |
| | | agent | allow separation | |
| | | | between two layers of | |
| | | | glove | |
| 2. | 6.0-8.0 | Pearlescent agent | Provide intensive silky | Perlogen SF 3000, Tego Pearl N 300, |
| | | | shine and smoothness to | EGMS, EGDS, Glycol Distearate |
| | | | glove surface | |
| 3. | 4.0-6.0 | Cationic silicone | Act as conditioning | Xiameter mem 0939, mem 0949, |
| | | emulsion | agent to provide smooth | MEM 8663, MEM 0346, OFX 5103 |
| | | | finishing on the glove | |
| | | | surface | |
| 4. | 4.0-6.0 | Mild surfactant | Conditioning glove | CS 98, CAB, Surfynol 465, Surfynol 440, |
| | | | surface between discrete | ACU 619, Triton X-100 |
| | | | layers | |
| 5. | 1.5-2.5 | Water and oil | Provide blooming effect | Olivem 300, Imwitor PG3 C10, |
| | | soluble functional | on the first latex layer to | Glyceryl oleate, Softigen 767, |
| | | lipid | produce discrete layers | Tegosoft PC 31, Tegosoft GC, |
| | | | | Oleamuls WS |
| 6. | 0.5-1.5 | Anti-microbial | Preserves the solution | Nipaguard SCP, Bacfree, |
| | | agent | from micro-organisms | Protectol, Nipaguard SCE |

TABLE G
Off-line Chlorination

| No. | Concentration, % | Material | Functionality | Examples of Material |
|---|---|---|---|---|
| 1. | 3.7 ± 0.2 | Chlorine | Reacts with hydrochloric acid | Sodium Hypochlorite |
| | | | to smoothen glove surface - | |
| | | | reduce tackiness | |
| 2. | 4.2 ± 0.2 | Acid | Reacts with sodium hypochlorite | Hydrochloric acid |
| | | | to smoothen glove surface - | |
| | | | reduce tackiness | |
| 3. | 4.0-6.0 | Neutralizer | Neutralizes the acidized glove | Ammonia |
| | | | surface | |

TABLE H
Off-line Polymer Coating-CS33

| | Parts by | | | |
|---|---|---|---|---|
| No. | weight, % | Material | Functionality | Examples of Material |
| 1. | 20-30 | Polyethylene wax | Provide slip, lubrication, | Aquacer 1030, BASF A, Honeywell AC6A, |
| | | additive | abrasion resistance and | Sasol H1, SN 118W |
| | | | anti-blocking properties to | |
| | | | the glove | |
| 2. | 30-40 | Silicone emulsion | Reduce coefficient friction | Xiameter MEM 0039, MEM 0349, |
| | | | and impart gloss on the glove | MEM 1785, MEM1171 |
| | | | surface | |
| 3. | 15-25 | Cationic silicone | Act as conditioning agent to | Xiameter 0939, Xiameter 0949, |
| | | emulsion | provide smooth finishing on | MEM 8663, MEM 0346, OFX 5103 |
| | | | the glove surface | |
| 4. | 15-25 | Mild surfactant | Conditioning glove surface | CS 98/CAB Surfynol 465, Surfynol 440, |
| | | | providing damp hand donning | ACU 619, Triton X-100 |
| | | | effect | |
| 5. | 0.5-1.5 | Stabilizer | Maintain pH of solution at | Ammonia, Potassium Hydroxide |
| | | | required specification to | |
| | | | prevent micro-organism growth | |
| | | | and solution destabilization | |

TABLE I
Offline surface spraying polymer

| No. | Concentration, % | Material | Functionality | Examples of Material |
|---|---|---|---|---|
| 1 | 4.4-4.8 | Offline Surface | Smoothen and eliminate | FU/CS 11, FU/CS 51, FU/CS 52, |
| | | Lubricant | tackiness of glove surface | SM 25, SM 26 |

### Example 3

### Process for the Production of a Multi-Layered Polymer Product

### Dipping Process Description Based on Lab Scale Dipping for Three Latex Dipping

Dipping process of this a Multi-Layered Polymer Product which is a surgical glove starts with former cleaning and drying. Drying process is done by heating the formers in oven at 60-70° C. In the meantime, coagulant mix is heated to 55±2° C. Completely dried formers are dipped into warm first coagulant (preferred former temperature is 55-60° C.) and dried in oven at 100° C. for 15-20 minutes. Coagulant coated and dried formers are then dipped into latex compound. This process comprises of three latex tanks in which two comprise white latex compounds, while the remaining tank comprises green PI latex compound. The tanks comprising the white compounds are of 28-30% TSC, while the tanks comprising the green compound is of 35-37% TSC. Coagulant coated formers are dipped into latex tank 1 followed by drying in oven at 100° C. for 2 min and subsequently dipped into latex tank 2. After latex tank 2 dipping, followed by 2 minutes drying in oven at 100° C. Then, latex coated formers are leached in hot water (60±2° C.) to remove excess residual chemicals and surfactant. Leached formers are air dried for one minute and dipped into a first on-line polymer compound (Dip PUD). Dip PUD dipping level is controlled at the level from fingertip to wrist. Dip PUD requires 10-15 minutes of drying in oven at 100° C. Sufficiently dried formers are dipped into anti-tack solution coating. Anti-tack solution dipping level is maintained at same level as Dip PUD. Anti tack dipped formers are then dried in oven at 100° C. for 2 minutes. Anti-tack solution coating dries quite fast compared to polymer solution. After that, the formers are dipped into coagulant 2 at full length from fingertip to cuff. Second coagulant coated formers requires 8 minutes of drying in oven at 100° C. Next, third layer of latex compound (green) is dipped. It requires only single layer coating. Then, second leaching, beading and a second on-line polymer compound (Dip 95) coating is conducted. Finally, the formers completely coated with three layers of latex are cured in vulcanizing oven at 125° C. for 30 minutes. After completion of vulcanisation gloves are stripped out from the formers. Stripped gloves ware post tumbled at 60-90° C. for 50-80 minutes.

### Dipping Process Description Based on Lab Scale Dipping for Two Latex Dipping

Dipping process of another Multi-Layered Polymer Product which is a glove starts with former cleaning and drying. Drying process is done by heating the formers in oven at 60-70° C. In the meantime, coagulant mix is heated to 55±2° C. Completely dried formers are dipped into warm coagulant 1 mix (preferred former temperature is 55-60° C.) and dried in oven at 100° C. for 15-20 minutes. Coagulant coated and dried formers are then dipped into latex compound. This process consists of two latex tanks, one white and one green PI latex compounds. Both white and green compounds are of 35-37% TSC. Coagulant coated formers are dipped into latex tank 1 and dried in oven at 100° C. for two minutes. Then, latex coated formers are leached in hot water (60±2° C.) to remove excess residual chemicals and surfactant. Leached formers are air dried for one minute and dipped into polymer 1, Dip PUD from fingertip to wrist. Dip PUD requires 10-15 minutes of drying in oven at 100° C. Sufficiently dried formers are dipped into anti-tack solution, also from fingertip to wrist and dried in oven at 100° C. for another two minutes. Anti-tack solution coating dries quite fast compared to polymer solution. After that, the formers are dipped into coagulant 2 at full length from fingertip to cuff. Coagulant 2 requires 8 minutes of drying in oven at 100° C. Coagulant coated formers are then dipped into latex tank 2 (green compound). Then, leaching followed by beading and subsequently dipped into polymer 2 (Dip 95). Finally the formers completely coated with layers of latex are cured in vulcanizing oven at 125° C. for 30 minutes. After completion of vulcanisation gloves are stripped out from the formers. Stripped gloves are post tumbled at 60-90° C. for 50-80 minutes.

### Offline Process for Unified Double Layer Polyisoprene Surgical Gloves

Post tumbled gloves are conditioned at room temperature for a duration of not less than 48 hours before transferring them to offline facility. Offline process starts with chlorination process. Post chlorination gloves are rinsed with plain water three times to remove excess chlorine solution. Once rinsing complete, glove is free tumbled (without elevated temperature) to remove excess water before rinsing them with an off-line polymer coating (CS33). This process will require 20 to 45 minutes of rinsing. Then, the gloves are dried in tumbler dryer at 55-65° C. for 75-105 minutes. Dried gloves are sprayed with grip adjusting polymer solution and continuously dried again for 45 to 60 minutes at 50-60° C. Finally, the gloves are cooled for 10 to 20 minutes in the tumbler.

Although the present invention has been described in a specific embodiment as in the above description, it is understood that the above description does not limit the invention to the above given details. It is apparent to those skilled in the art that various changes and modification may be made therein without departing from the principle of the invention or from the scope of the appended claims.

### REFERENCE LIST

Multi-Layered Polymer Product (1)
Outer Layer (2)
Inner Layer (3)
Main Body (4)
Secondary Body (5)

1. A multi-layered polymer product (1) comprising a main body (4) and a secondary body (5), characterized in that said main body (4) and secondary body (5) comprise:
i. an outer layer (2);
ii. one or a plurality of intermediate layers; and
iii. an inner layer (3):
wherein the outer (2), intermediate and inner (3) layers of the main body (4) are attached at a region as to form discrete layers, while the outer (2), intermediate and inner (3) layers of the secondary body (5) are attached together entirely.

2. The multi-layered polymer product (1) according to claim 1, wherein the intermediate layers of the main body (4) comprise an anti-tack solution coating and a polymer coating.

3. The multi-layered polymer product (1) according to claim 1, wherein said anti-tack solution coating comprises
i. an anti-caking agent;
ii. a pearlescent agent;
iii. a conditioning agent;
iv. a surfactant;
v. a functional lipid; and,
vi. polyglyceryl ester.

4. The multi-layered polymer product (1) according to claim 3, wherein said anti-caking agent comprises soap, detergent, stearate, or a combination thereof.

5. The multi-layered polymer product (1) according to claim 3, wherein said conditioning agent is an emulsion.

6. The multi-layered polymer product (1) according to claim 3, wherein said surfactant comprises anionic surfactant, cationic surfactant or a combination thereof.

7. The multi-layered polymer product (1) according to claim 2, wherein said polymer coating comprises
i. a binder;
ii. a stabilizer;
iii. a silicone;
iv. a wax emulsion;
v. a matting agent;
vi. a dispersing agent;
vii. a wetting agent; and,
viii. a coupling agent.

8. The multi-layered polymer product (1) according to claim 7, wherein said binder comprises a soft binder, an acrylic binder, a medium hard binder, a styrene acrylic binder or a combination thereof.

9. The multi-layered polymer product (1) according to claim 7, wherein said silicone comprises a low molecular weight silicone, high molecular weight silicone or a combination thereof.

10. The multi-layered polymer product (1) according to claim 1, comprising a polymer selected from polyisoprene-polyisoprene latex, polyisoprene-chloroprene latex, polyisoprene-nitrile butadiene latex, polyisoprene-chloroprene-nitrile butadiene latex, or a combination thereof.

11. The multi-layered polymer product (1) according to claim 1, wherein said product comprises gloves, condoms, shower caps or any polymer product which requires donning.

12. A method for producing a multi-layered polymer product (1) comprising the steps of:
i. providing a former having a shape of a desired product;
ii. dipping said former in a first coagulant, to a preset immersion depth t₁;
iii. dipping said former in a first latex dispersion to the immersion depth t₁ to produce an inner layer;
iv. dipping said former in a second latex dispersion to the immersion depth t₁ to produce at least one intermediate layer;
v. dipping said former in a polymer coating to a preset immersion depth t₂;
vi. dipping said former in an anti-tack solution coating to the immersion depth t₂;
vii. dipping said former in a second coagulant to the immersion depth t₁;
viii. dipping said former in the third latex layer to the immersion depth t₁ to produce an outer layer; and,
ix. dipping said former in a second polymer coating to a preset immersion depth t₃.

13. The method according to claim 12, wherein said latex layer immersion depth t₂ and anti-tack solution coating produces said outer (2), intermediate and inner (3) latex layers that are attached at a region producing a main body (4).

14. The method according to claim 12, wherein said latex layer immersion depth t₁ produces said outer (2), intermediate and inner (3) latex layers that are attached together entirely producing a secondary body (5).

15. The method according to claim 12, comprising a vulcanization step which is carried out at a temperature between 80° C. to 120° C., at approximately 100° C., on the outer (2), intermediate and inner (3) latex layers.

16. The method according to claim 12 for producing a glove, raincoat, condom, shower cap or any polymer products that requires donning.

17. The method according to claim 12, wherein said polymer coating comprises
i. a binder;
ii. a stabilizer;
iii. a silicone;
iv. a wax emulsion;
v. a matting agent;
vi. a dispersing agent;
vii. a wetting agent; and,
viii. a coupling agent.
